(19) United States
(12) Patent Application Publication
(10) Pub. No.: US 2016/0338992 A1
(43) Pub. Date: Nov. 24, 2016

(54) COMPOSITE COMPRISING 3'-HYDROXYGENISTEIN AND USE FOR INHIBITION OF MELANOGENESIS

(71) Applicants: BIO-JOURDENESS INTERNATIONAL GROUP CO., LTD., TAICHUNG CITY (TW); NATIONAL UNIVERSITY OF TAINAN, TAINAN CITY (TW)

(72) Inventors: TE-SHENG CHANG, TAINAN CITY (TW); WEI-MING TZENG, TAINAN CITY (TW)

(21) Appl. No.: 15/151,738

(22) Filed: May 11, 2016

(30) Foreign Application Priority Data
May 21, 2015 (TW) 104116192

(51) Int. Cl. A61K 31/353

(52) U.S. Cl. CPC A61K 31/353

(57) ABSTRACT

A composite comprising 3′-hydroxygenistein and a use for inhibition of melanogenesis are revealed herein. An effective dose of 3′-hydroxygenistein is applied to skin for inhibition of tyrosinase activity and further inhibition of melanogensis, so that 3′-hydroxygenistein can be used in cosmetic composition.

### BACKGROUND OF THE INVENTION

1. Field of the Invention

The present invention relates to a composite comprising 3′-hydroxygenistein and use of the same for inhibition of melanogenesis, especially to a cosmetic composition of 3′-hydroxygenistein used for skin whitening due to its inhibitory effect on tyrosinase activity and melanogenesis.

2. Descriptions of Related Art

Human skin color is mainly determined by distribution of melanin in the epidermis of the skin. The formation of melanin, through a process called melanogenesis, occurs in melanosomes of the melanocytes (pigment-producing cells) for against ultraviolet light. The melanogenesis consists of a combination of enzyme-catalyzed chemical reactions. Tyrosinase is one of the key enzymes in melanogenesis, not only involved in a plurality of reactions in melanin synthesis, but also playing a key role in the rate-limiting step catalyzed by the enzyme. Thus the tyrosinase content can be used as an indicator of melanogenesis. Although melanin has a photoprotective function, excess melanin production and abnormal distribution can cause irregular hyperpigmentation and the appearance of dark spots on the skin. Thus how to inhibit tyrosinase activity effectively for prevention of hyperpigmentation such as melasma and aged spots have become key points of the research.

Flavonoids, such as daidzein, apigenin, genistein, liquiritigenin, naringenin, catechin, etc. are a class of important secondary metabolites of plants. In recent years, the physiological properties of flavonoids including antioxidant, prevention of osteoporosis, reduction of cardiovascular diseases and alleviating the menopausal syndromes have been found. For example, apigenin and genistein have shown significant effects on antioxidant, anti-inflammatory and protection from photodamage (Photochem Photobiol. 2008 March-April; 84(2):489-500). Some research also has proven that bioactivities of flavonoids are also affected by functional groups on different carbon atoms of the carbon skeleton thereof Especially hydroxyl flavonoid derivatives have bioactivities quite different from the precursor-flavonoids. The past research of the inventor of the present invention has proven ortho-hydroxydaidzein (OHDe) derivatives including 6-hydroxydaidzein (6,7,4′-trihrdroxyisoflavone; 6-OHDe, Biosci. Biotechnol. Biochem. 2005, 69(10), 1999-2001) and 8-hydroxydaidzein (7,8,4′-trihrdroxyisoflavone, 8-OHDe, J. Agric. Food Chem. 2007, 55(5), 2010-2015; Int. J. Mol. Sci. 2009, 10(10), 4257-4266) both have tyrosinase inhibition activity but its precursor-daidzein doesn't have such activity. Moreover, refer to Taiwanese Pat. No. 1312686 “melanin inhibitor”, it is revealed that 6-OHDe, 8-OHDe and 8-hydroxygenistein (5,7,8,4′-tetrahydroxyisoflavone) have the tyrosinase inhibitory activity that is 25 times stronger than that of glycitein, 30 times stronger than that of daidzein and 90 times stronger than that of genistein. Therefore the bioactivities of hydroxyl flavonoids are significantly different from those of its precursor flavonoids.

Whether 3′-hydroxygenistein exerts inhibitory effect on tyrosinase activity hasn't been confirmed yet. There is room for improvement and a need to find out more flavonoid derivatives that inhibit melanogenesis. Moreover, the derivatives are applied to development of a low-dose skin care product with high efficiency.

### SUMMARY OF THE INVENTION

Therefore it is a primary object of the present invention to provide a composite comprising 3′-hydroxygenistein and use of the same for inhibition of melanogenesis, wherein the 3′-hydroxygenistein is represented by the following structural formula:

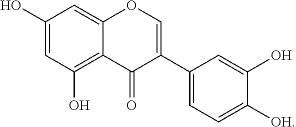 [SMILES: O=c1c(-c2ccc(O)c(O)c2)coc2cc(O)cc(O)c12]

The 3′-hydroxygenistein is used in a cosmetic composition for skin whitening and spot treatment due to its inhibitory effect on tyrosinase activity and melanogenesis.

In order to achieve the above object, 3′-hydroxygenistein according to the present invention is used in composite that inhibits melanogenesis. An effective dose of 3′-hydroxygenistein is applied to skin for inhibition of tyrosinase activity and further inhibition of melanogenesis. The effective dose is ranging from 10 μM to 40 μM while 10 μM to 20 μM is preferred.

Thereby 3′-hydroxygenistein according to the present invention is further used in cosmetic composite for skin whitening such as water base cosmetics, emulsion cosmetics, ointment base cosmetics, power cosmetics, etc. 3′-hydroxygenistein can also be added into other functional cosmetics such as humectants, or anti-wrinkle cosmetics.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein:

FIG. 1 shows an embodiment of 3′-hydroxygenistein obtained from biotransformation according to the present invention;

FIG. 2 shows effect of 3′-hydroxygenistein under different treatment conditions on tyrosine activity according to the present invention;

FIG. 3 shows cytotoxicity of 3′-hydroxygenistein according to the present invention;

FIG. 4 shows effect of 3′-hydroxygenistein under different treatment conditions on melanogenesis according to the present invention.

### DETAILED DESCRIPTION OF THE PREFERRED EMBODIMENT

3′-hydroxygenistein is used in preparation of composite that inhibits melanogenesis. An effective dose of 3′-hydroxygenistein is applied to skin for inhibition of tyrosinase activity and further inhibition of melanogenesis. The effective dose is ranging from 10 μM to 40 μM while 10 μM to 20 μM is preferred.

It should be noted that there are a plurality of ways for preparation of 3′-hydroxygenistein. There is no limit on the preparation way.

### Experiment 1: Preparation of 3′-hydroxygenistein

In an embodiment of the present invention, 3′-hydroxygenistein is prepared by the following biotransformation.

(1) Construction of Recombinant Pichia pastoris containing a Fusion Gene for Biotransformation of Flavonoids

A recombinant plasmid containing CYP57B3 gene from Aspergillus oryzae and a cytochrome reductase gene (CPR) from Saccharomyces cerevisiae is transformed into microbial expression system, Pichia pastoris for expression and obtaining 3′-hydroxygenistein. The technique to construct recombinant P. pastoris including CYP57B3 gene and CPR gene is well-known to the people skilled in the art.

(2) Fermentation and Preparation of 3′-hydroxygenistein

Recombinant Pichia pastoris is cultured in 100 mL Yeast Nitrogen Base (YNB) (containing 2% dextrose and 100 μg/ml Zeocin) at 28° C. for 48 hours with shaking at 200 rpm to get a seed culture. Then the seed culture is inoculated into a 5 L fermentor containing 2.5 L YNB, 2% dextrose, 250 μM δ-aminolevulinic acid, and 100 μM genistein. Fermentation is conducted at 28° C. with agitation 280 rpm and 0.5 v/v/m (volume of air/volume of medium/minute) aeration for 72 hours.

### (3) Analysis of Filtrate by Ultra Performance Liquid Chromatography (UPLC)

After fermentation, the amount of genistein and 3′-hydroxygenistein produced in cultures are analyzed and determined by using UPLC. For gradient elution, the C18 reversed-phase column (Acquity UPLC BEH C18, 1.7 μm, 2.1 i.d.×100 mm, Waters, USA), 1% (v/v) acetic acid in water (solution A) and methanol (solution B) are used. The column is eluted by a linear gradient from 15% to 35% solution B for 5 minutes with the flow rate of 0.3 ml/min and absorbance measured at 260 nm. According to the standard curve obtained by measuring of multiple standards, the 3′-hydroxygenistein content produced is calculated.

### (4) Isolation and Identification of Biotransformation Products by High-Performance Liquid Chromatography (HPLC)

Prepare four batches of 2.5 L fermentation broth for purification of biotransformation products. After fermentation, use 2 L ethyl acetate to extract the broth twice. Then all the ethyl acetate extract containing 3′-hydroxygenistein is concentrated by vacuum concentration to form a residue. The residue is dissolved in 400 ml 50% methanol and then filtered and purified by a 2.2 μm nylon membrane filter to get a filtrate. Perform high-performance liquid chromatography (HPLC) by using preparative C18 reversed-phase column (Inertsil, 10 μm, 20.0 i.d.×250 mm, ODS 3, GL Sciences, Japan). The solution A and solution B are used for gradient elution. The column is eluted by a linear gradient from 25% to 50% solution B for 25 minutes with the flow rate of 15 ml/min, ultraviolet detection at 260 nm and injection volume of 10 ml. Collect elution corresponding to the peak in UPLC, vacuum concentrate the elution and freeze dry to get 11.5 mg crystals. At last, use nuclear magnetic resonances (NMR) and mass spectrometry to determine chemical structure of the crystals.

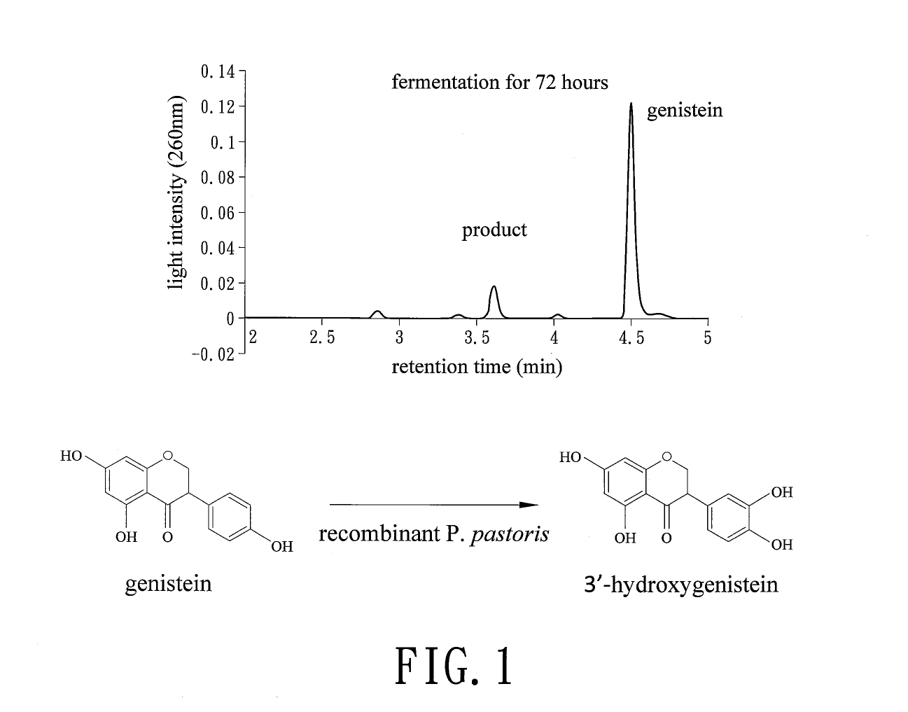

Refer to FIG. 1, a peak of the product resulting from biotransformation of genistein by recombinant Pichia pastoris in the fermentor containing genistein for 72 hours appear at the retention time of 3.6 min in the ultra performance liquid chromatography (UPLC). The product is further isolated by using HPLC and then identified by NMR and mass spectrometry. The data obtained are as follows, ESI/MS m/z: 295 [M−H]⁺; ¹H-NMR (DMSO-d6, 500 MHz) δ: 6.20 (1H, d, J=1.8 Hz, H-6), 6.36 (1H, d, J=1.8 Hz, H-8), 6.76 (1H, d, J=8.0 Hz, H-5′), 6.78 (1H, dd, J=8.0, 1.8 Hz, H-6′), 6.96 (1H, d, J=1.8 Hz, H-2′), 8.22 (1H, s, H-2); ¹³C-NMR (DMSO-d6, 125 MHz) 6:180.6 (C-4, C═O), 164.7 (C-7), 162.3 (C-5), 157.9 (C-9), 154.3 (C-2), 145.8 (C-3′), 145.2 (C-4′), 122.8 (C-1′), 122.0 (C-3), 120.4 (C-6′), 116.8 (C-5′), 115.8 (C-2′), 104.8 (C-10), 99.4 (C-6), 94.1 (C-8). Compared with data reported in the literature, it is learned that the product is 3′-hydroxygenistein. Later the 3′-hydroxygenistein obtained by biotransformation is tested.

### Experiment 2: Detection of the Effect of 3′-hydroxygenistein on Tyrosinase

Tyrosinase plays an important role on melanogenesis. Thus the effect of 3′-hydroxygenistein on tyrosinase is detected. First dissolve 1 μL 3′-hydroxygenistein in DMSO (dimethyl sulfoxide) and mixed with 5 μL tyrosinase in a 94 μL, 50 mM PBS (pH 6.8). Add 0.9 mL, 2.5 mM L-DOPA (dissolved in 50 mM PBS, pH 6.8) into the mixture and react at 25° C. for 10 minutes. The formation of dopachrome is determined by spectrophotometer ((U-5100; Hitachi, Japan) that detects absorbance at 475 nm. The activity of tyrosinase is represented by percentage of the DMSO control group. In this experiment, kojic acid and genistein are used as positive controls.

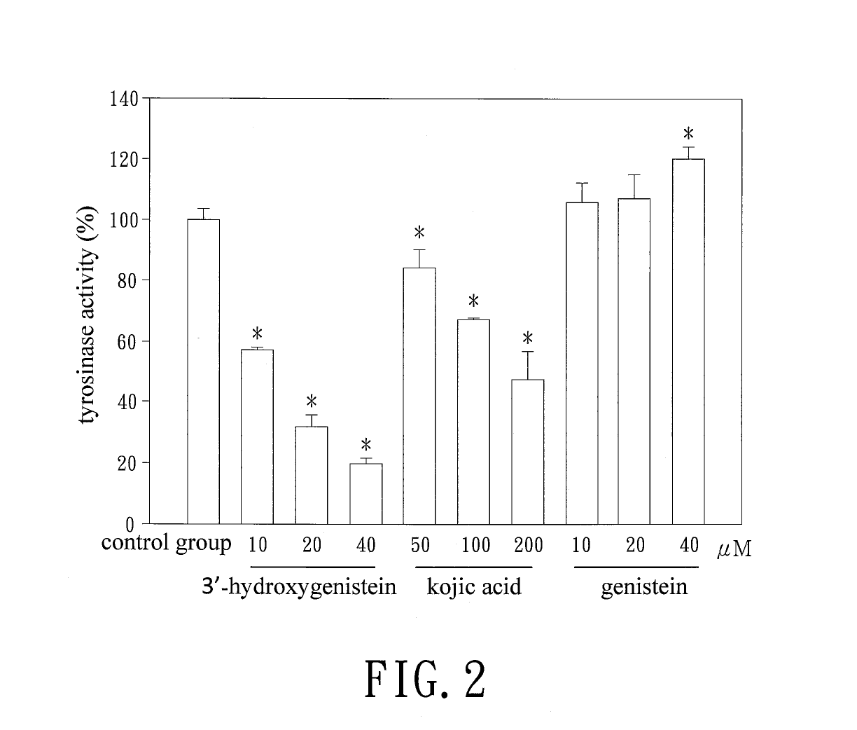

Refer to FIG. 2, the Y axis represents the tyrosinase activity while the X axis includes different treatment conditions. The results show that 10-40 μM 3′-hydroxygenistein inhibits tyrosinase activity in a dose-dependent manner The higher the dose, the better the inhibitory effect. Moreover, the half-inhibitory concentration (IC₅₀) of 3′-hydroxygenistein in inhibiting tyrosinase is 15.9 μM. The 3′-hydroxygenistein has the tyrosinase inhibitory activity (IC₅₀ 15.9 μM) that is 11 times stronger than that of kojic acid (IC₅₀ 183.6 μM) used as the standard. It is also learned that genistein at the same concentration shows no tyrosinase inhibitory activity.

### Experiment 3: Cytotoxicity of 3′-hydroxygenistein and effect of 3′-hydroxygenistein on melanogenesis

### (1) Detection of Cell Viability

In order to confirm a non-cytotoxic concentration range of 3′-hydroxygenistein, MTT and crystal violet staining assays are used to detect cell viability of 3′-hydroxygenistein. A mouse melanoma cell line (B16 melanoma 4A5 obtained from bioresource Collection and Research Center, Food Industry Research and Development Institute) is cultured in DMEM (Dulbecco's Modified Eagle Medium) containing 10% (v/v) fetal bovine serum (FBS) in a 5% CO₂ humidified incubator for 24 hours. The B16 melanoma 4A5 cells are treated with 3′-hydroxygenistein for 48 hours. Then remove the culture medium, add 1 mg/mL MTT dissolved in PBS and incubate for 2 hours. Next remove MTT solution and add DMSO. At last, use spectrophotometer ((U-5100; Hitachi, Japan) to detect absorbance of dissolved formazan crystals at 475 nm. The cell viability is expressed as percentage of control (% of control).

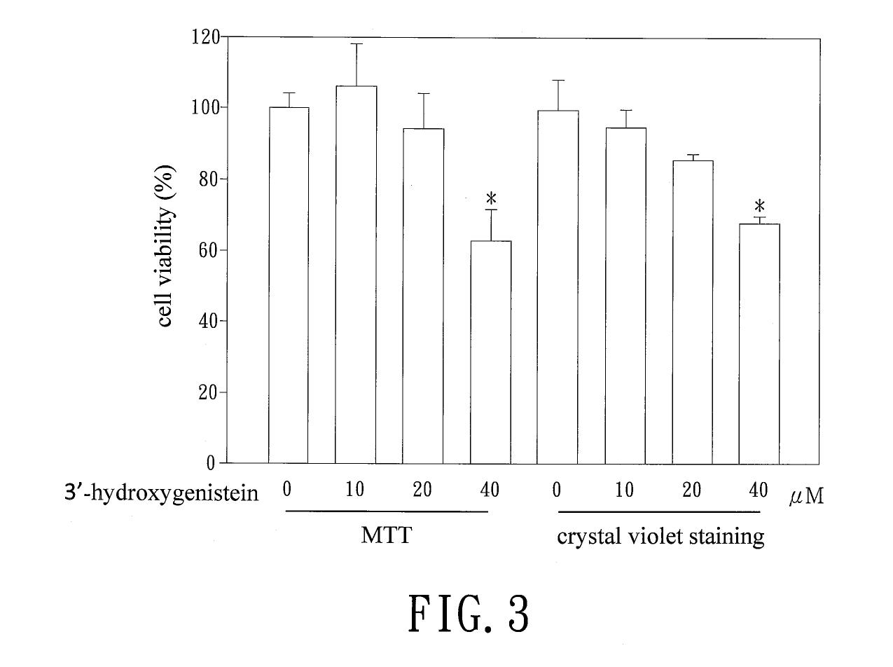

As shown in FIG. 3, the Y axis of the results represents cell survival rate and the X axis includes different treatment conditions. Compared with the control group (0 μM), 3′-hydroxygenistein with concentration between 10 μM and 20 μM has no cytotoxicity while 40 μM 3′-hydroxygenistein shows cytotoxicity. Thus the following experiment only discusses inhibitory effects of 20 μM 3′-hydroxygenistein on melanin

### (2) Detection of Melanin Content

In this experiment, 3-isobutyl-1-methylxanthine (IBMX) is used as a melanogenic-stimulating agent while genistein and danazol with inhibitory effect on melanogenesis are used as positive controls. The B 16 melanoma 4A5 cells are cultured in DMEM containing 10% (v/v) fetal bovine serum (FBS) in a 5% CO₂ humidified incubator at 37° C. Then seed cells in a 24-well plate with proper density. After one day, the cells are treated with 5 μM, 10 μM, and 20 μM 3′-hydroxygenistein respectively in the presence of 400 μM IBMX and cultured for 2 days. Then collect and wash the cells with PBS (Phosphate Buffered Saline) buffer twice. The cell pellets are dissolved in lysis buffer containing 20 mM trisodium phosphate (pH6.8) and 1% Triton X-100. After centrifugation at 15,000×g for 15 minutes, melanin pellets are dissolved in 1N NaOH containing 20% DMSO at 95° C. for 1 hour. Use spectrophotometer ((U-5100; Hitachi, Japan) to detect absorbance of melanin at 490 nm. The melanin content is expressed as percentage of the control group (IBMX group).

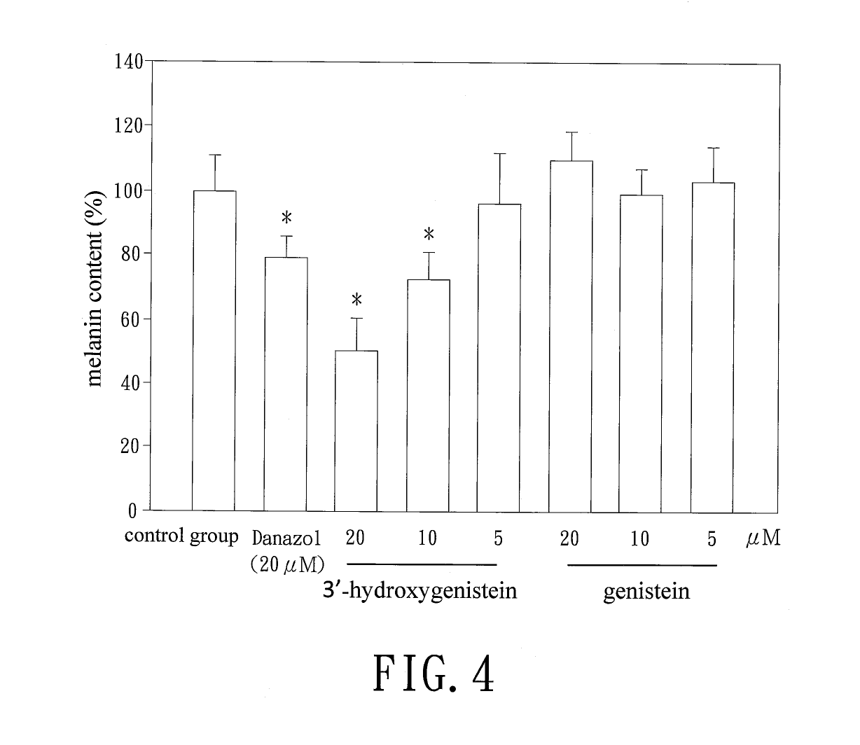

Refer to FIG. 4, the results are shown while the Y axis represents melanin content and the X axis includes different treatment conditions. Compared with the control group, 3′-hydroxygenistein with concentration of 10 μM and 20 μM show inhibition of melanogenesis in B16 melanoma 4A5 cells, especially the melanin content of the group treated with 20 μM 3′-hydroxygenistein is decreased to 50.5%. The results also show that inhibitory effect of 3′-hydroxygenistein on melanogenesis is better than danazol which has been proven to be a potent melanogenesis inhibitor (Arch Pharm Res 2010; 33:1959-1965). It can also be learned from FIG. 4 that the genistein at the same concentration shows no inhibitory effect on melanogenesis.

In summary, 3′-hydroxygenistein do have inhibitory effect on tyrosinase and melanogenesis. Thereby 3′-hydroxygenistein can be used as material for whitening cosmetics such as water base cosmetics, emulsion cosmetics, ointment base cosmetics, power cosmetics, etc. The cosmetic composition further includes at least one commonly-used cosmetically acceptable adjuvant selected from solvents, gelling agents, active agents, preservatives, antioxidants, screening agent, chelating agents, surfactants, thickening agent, perfumes, and odor absorbers.

The cosmetic composition can also be used together with at least one external use agent selected from whitening agents, humectants, anti-inflammatory agents, ultraviolet absorbers, plant extracts, anti-acne agents, antipsoriatic agents, antiagers, antiwrinkle agents and wound-healing agents.

Compared with the technique available now, the present invention has the following advantages:
1. The present invention has proven that 3′-hydroxygenistein inhibits tyrosinase activity and melanogenesis for the first time. Thus 3′-hydroxygenistein is a novel flavonoid used in cosmetic composition.
2. The present invention has also proven that a low amount of 3′-hydroxygenistein (10 μM˜20 μM) has optimal inhibitory effect on tyrosinase activity and melanogenesis. The inhibitory effect of 3′-hydroxygenistein on melanogenesis is significantly higher than kojic acid (tyrosinase inhibitor) and danazol (melanogenesis inhibitor) while genistein at the same concentration has not inhibitory effect on tyrosinase activity and melanogenesis. The literature has reported that genistein at concentrations ranging from 30 μM to 60 μM can induce melanin cells to produce more melanin (J Nutr Biochem 2002; 13:421-426). The 3′-hydroxygenistein is highly effective in skin whitening at low dose.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalents.

What is claimed is:

1. A method for inhibiting melanogenesis by composites containing 3′-hydroxygenistein comprising the step of:
applying the composites containing an effective dose of 3′-hydroxygenistein to skin so as to decrease tyrosinase activity and further inhibit melanin synthesis;
wherein the 3′-hydroxygenistein is represented by the following structural formula:

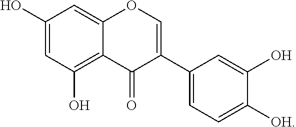 [SMILES: O=c1c(-c2ccc(O)c(O)c2)coc2cc(O)cc(O)c12]

2. The method as claimed in claim 1, wherein the effective dose is ranging from 10 μM to 40 μM.

3. The method as claimed in claim 2, wherein the effective dose is ranging from 10 μM to 20 μM.

4. The method as claimed in claim 1, wherein the composite is in the form of cosmetic composition.

5. The method as claimed in claim 4, wherein the cosmetic composition is selected from the group consisting of water base cosmetics, emulsion cosmetics, ointment base cosmetics, and power cosmetics.
